(19) United States
(12) Patent Application Publication
(10) Pub. No.: US 2006/0233862 A1
(43) Pub. Date: Oct. 19, 2006

(54) Process for the preparation of a fat composition containing sterol esters a product obtained by said process and the use thereof

(75) Inventor: Jari Alander, Karlshamn (SE)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: KARLSHAMNS AB, Karlshamn (SE)

(21) Appl. No.: 11/356,249

(22) Filed: Feb. 17, 2006

Related U.S. Application Data
(63) Continuation of application No. 10/451,784, filed on Jul. 11, 2003, now abandoned.

(30) Foreign Application Priority Data
Jan. 11, 2001 (SE) 0100080-1

(51) Int. Cl. A61K 47/00; C07J 9/00; A61K 36/889; A61K 36/899; A61K 36/31; A61K 36/48; A61K 36/28; A61K 36/55

(52) U.S. Cl. 424/439; 552/540; 424/768; 424/776; 424/727; 424/755; 424/750; 424/757; 424/764

(57) ABSTRACT

A method for preparing a fat composition by reacting a sterol raw material with a triglyceride raw material to create the fat composition.

The present invention refers to a new process for the preparation of a sterol ester containing fat composition, to the product obtained by said process, and to the use thereof.

### BACKGROUND

Lowered serum cholesterol levels in humans is desirable since it is associated with a lower risk for cardiac disease and atherosclerosis. It is well known that dietary phytosterols reduce serum cholesterol by inhibiting cholesterol absorption. Phytosterols are in this context defined as sterols and sterol derivatives found in and extracted from various types of plant materials, with beta-sitosterol and its esters as the most abundant and well-known representatives.

Free, that is unesterified, sterols have melting points in excess of 100° C., depending on actual chemical structure, and a low solubility in vegetable oils and an extremely low solubility in water. This combination of low oil solubility and high melting point will give compositions containing free sterols a poor palatability and brings about difficulties in modern high-speed food manufacturing processes. The poor solubility in aqueous solutions, such as the body fluids, hampers the bioavailability and thus the cholesterol lowering ability of said sterols.

In order to obtain an improved biological uptake it is today customary to use esters of phytosterols. Miettinen, T. A., et al., (1995), Reduction of serum cholesterol with sitostanol-ester margarine in a mildly hypercholesterolemic population, N. Engl. J. Med. 333, 1308-1312, has shown that a daily intake of 1.8 or 2.6 grams of sitostanol in the form of ester decreased the serum total and LDL cholesterol concentrations by about 10 to 14% in subjects with mild hypercholesterolemia. In another study a similar reducing effect on total and LDL-cholesterol concentrations, that is 8 and 13%, respectively, was obtained in healthy adults after an intake of a fat spreadenriched with soybean oil sterols, primarily esters of sitosterol, campesterol and stigmasterol, in an amount of about 3 g per day, see Weststrate, J. A. et al., (1998), Plant sterol-enriched margarines and reduction of plasma total- and LDL-cholesterol concentrations in normocholesterolaemic and mildly hypercholesterolaemic subjects, Eur. J. Clin. Nutr. 52, 334-343.

### PRIOR ART

Hydrophobic sterol esters have good solubility in vegetable oils and fats. The esters occur naturally in small amounts in most vegetable oils and fats and consist generally of a long chain fatty acid ester of des-methyl, monomethyl- and dimethylsterols. Some materials contain relatively high amounts of esters of phenolic acids and triterpene alcohols, for example shea butter, rice bran oil and corn fiber oil.

Synthetic esters of free phytosterols can be obtained by direct esterification or by inter- or in another word transesterification.

Direct esterification of sterols and stanols with fatty acids using sodium bisulphate as catalyst is described in U.S. Pat. No. 5,892,068. The exemplified acid catalyzed reactions were carried out at a temperature of 150° C. for 16 hours forming discrete stanol/sterol-esters which were then isolated from the reaction mixture. This process, however, requires that the sterol or stanol used as a starting material is available in purified form and also that the resulting ester product is isolated.

A conventional way to esterify sterols is by interesterification with a fatty acid ester of a low-boiling monohydric alcohol. The fatty acid ester used as the starting material is prepared by reacting an alcohol, that is in general methanol, with a vegetable oil such as canola oil or sunflower oil using an alkaline catalyst, such as sodium hydroxide or sodium methoxide. Glycerol is split off and recovered, and the excess of monohydric alcohol, that is methanol, is evaporated and recirculated. The resulting methyl ester is washed and dried, and subsequently reacted with the sterol in the interesterification described, releasing methanol which is evaporated and giving a mixture of excess methyl esters and sterol esters. Similar processes can be performed with other low-boiling alcohols such as ethanol or isopropanol, but methanol is the conventional choice. According to WO 99/19640 a pure β-sitostanol was esterified with a rapeseed oil methyl ester mixture by heating a mixture thereof at 90-120° C. under a vacuum of 5-15 mm Hg, adding Na-ethylate and continuing the reaction giving an ester mixture which could be used as such as an edible additive in fats. GB 1 405 346 refers to a process for the conversion of sterols with free hydroxyl groups, naturally contained in vegetable or animal oils and fats, into their fatty acid esters. The purpose of this esterification was to protect the sterols against degradation during bleaching and hardening in the course of refining of the fat or oil. The conversion into fatty acid esters takes place by mixing the sterol containing oil with 1.0-1.1 equivalents, relative to the free sterol content, of fatty acid esters of monohydric aliphatic alcohol with 1-4 C, and transesterifying the mixture at elevated temperatures in the presence of an alkali metal alcoholate or alkali metal as catalyst. The above mentioned processes require several steps and in addition toxic by-products are formed.

It should be mentioned that transesterification of edible fats and oils is a common procedure which is carried out without any other additives than catalysts in order to amend the properties of the fats or oils. During said process optionally present, free sterols are only partially converted into their fatty acid esters.

### DESCRIPTION OF THE INVENTION

It has now been found that a sterol ester containing fat composition can be made directly, without going through the extensive chemical reaction steps of ester interchange with a low-boiling alcohol component, in an industrially economical process.

A fat composition containing sterol esters can be obtained by drying a mixture of a triglyceride oil or fat and a sterol raw material until essentially free of water, carrying out an interesterification using alkaline catalysis and bleaching and deodorising the reaction mixture. The triglyceride is optionally pretreated by alkaline or physical refining, bleaching and deodorising in order to give a suitable starting material for the interesterification reaction.

The present invention refers to a process for the preparation of a fat composition containing sterol esters, characterised by direct interesterification of sterol with triglyceride in a one pot process giving a fat composition essentially containing sterols, fatty acid sterol esters and glycerides, which process comprises the following steps:

mixing a sterol raw material with a triglyceride raw material in a sterol to triglyceride ratio of 5/95 to 65/35 by weight,

heating the mixture to a temperature sufficient to partially or completely dissolve the sterol raw material in the triglyceride raw material and to reduce the water content thereof, optionally at a reduced pressure and under an inert atmosphere,

adding an alkaline catalyst in a catalytically effective amount,

allowing the interesterification reaction to take place,

neutralising the catalyst by the addition of acid, and finally

purifying the fat composition obtained, optionally after mixing with a food fat base.

The sterol raw material is selected from products obtained from the refining of vegetable oils or from the production of tall oil. The sterol material is normally a mixture of various individual sterols. Sterols derived from vegetable fats are normally dominated by beta-sitosterol, campesterol and stigmasterol. Each raw material has its typical sterol composition. For example, rapeseed sterols contain 15-30% brassicasterol, which substance is not abundant in other raw materials.

The sterol material derived from tall oil is also dominated by beta-sitosterol, especially if the tall oil sterol has been fractionated. Tall oil sterols also contain significant amounts of saturated sterols, also known as stanols.

4,4-dimethylsterols, also known as triterpene alcohols, is another class of phytosterols suitable for use in the present invention. Triterpene alcohols are found in large quantities in shea butter, rice bran oil, and corn fibre oil.

The native phytosterols can be hydrogenated completely or partially to yield stanols and sterol/stanol mixtures which can then be used within the scope of the present invention.

Preferred sterol raw materials according to this invention comprise tall oil sterols, completely or partially hydrogenated tall oil sterols, soybean, rapeseed (canola, lobra), and sunflower sterols, partially or completely hydrogenated soybean, rapeseed (canola, lobra), and sunflower sterols, or mixtures thereof.

According to a preferred aspect the invention refers to a process wherein the sterol raw material contains >90% by weight of one or more sterols selected from the group consisting of β-sitosterol, β-sitostanol, campesterol, campostanol, brassicasterol.

The triglyceride raw material can be any vegetable or animal oil or fat which can be used for food, cosmetic or pharmaceutical application. Depending on the physical and nutritional properties desired, different triglyceride raw materials are utilised. Examples of raw materials containing triglycerides that can be used in the interesterification process of the invention are the following: rapeseed oil (Brassica napus, rapa, campestris etc), crambe oil (Crambe abyssinica, hispanica), mustard seed oil (Brassica alba, hirta, nigra, juncea, carinata), soybean oil (Glycine max), sunflower oil (Helianthus annuus), cottonseed oil (Gossypium hirsutum, barbadense, herbaceum), peanut (or groundnut, or arachis) oil (Arachis hypogaea), linseed oil (Linus usitatissimum), evening primrose oil (Oenothera biennis, larmarkiana), borage oil (Borago officinalis), grapeseed oil (Vitis vinifera), safflower oil (Carthamus tinctorius), sesame oil (Sesamum indicum, orientate), tea seed oil (Thea sasanqua, Camellia sasanqua), corn (or maize) oil, corn fibre oil, corn bran oil (Zea mays), wheat oil, wheat bran oil or wheat germ oil (Triticum aestivum), oat oil, oat bran oil (Avena sativa), rice bran oil, rice oil (Oryza sativa), olive oil (Olea europea), palm oil, palm kernel oil (Elaeis guineensis, oleifera), coconut oil (Cocos nucifera), babassu oil (Orbignya martiana, oleifera), illipe butter, Borneo tallow (Shorea stenoptera), shea butter or shea oil (Butyrospemmum parkii) madhuca, mowrah butter (Madhuca latifolia, indica, longifolia), sal butter (Shorea robusta), mango seed oil (Mangifera indica), avocado oil, avocado seed oil (Persea americana), cocoa butter (Theobroma cacao), hazelnut oil (Corylus avellana), almond oil (Prunus amygdala), macadamia nut oil (Macadamia tetraphylla), walnut oil (Juglans nigra), and chestnut oil (Castanea mollissima), fish oils (menhaden, herring, tuna, salmon), tallow, lard, milk fat, butter fat.

Preferred examples of such oils are liquid standard oils, such as rapeseed oil, canola oil, corn oil, peanut oil, soybean oil, and sunflower oil, but also semi-solid oils, such as palm oil, and coconut oil, as well as mixtures thereof.

Another group of preferred oils are oils having specific fatty acids, such as borage oil, evening primrose oil, blackcurrant seed oil, fish oil, and linseed oil, as well as mixtures thereof.

According to another preferred aspect the invention refers to a process, wherein the triglyceride raw material comprises glycerolesters of saturated or unsaturated C4-C28 fatty acids, preferably C16-C22.

The invention especially refers to a process wherein the mixture of sterol raw material and triglyceride material is heated to a temperature 100-200° C., preferably 120-140° C., for a period of time being sufficient for essentially eliminating the water in the mixture. In a typical process, the refined and bleached triglyceride is mixed with the sterol powder, and heated under vacuum and stirring to 120-140° C. for 30-60 minutes to remove any residual water.

An alkaline catalyst such as sodium hydroxide, potassium hydroxide, sodium methoxide (sodium methylate), sodium ethoxide (sodium ethylate) or sodium glycerolate, is added to the reaction mixture and the interesterification reaction is carried out during 60-180 minutes. The amount of catalyst needed is dependent on the quality of the oil used and typically 0.1% based on the weight of the sterol/triglyceride mixture is sufficient. Preferably the alkaline catalyst is selected from the group consisting of NaOH, KOH, sodium methylate and sodium ethylate.

The interesterification reaction is allowed to take place for a period of 60-240 minutes at a temperature of 100-200° C., preferably at 120-140° C., that is at a temperature that is sufficiently high for the reaction to take place and sufficiently low not to decompose the oil. The reaction is preferably performed under non-oxidizing conditions, for instance under nitrogen. The reaction is then stopped by cooling the reaction mixture to below 100° C., adding water or an acid dissolved in water and precipitating an insoluble salt or collecting the neutralisation product on an adsorbent. The adsorbent can be any suitable amorphous silica used for removing polar compounds in vegetable oil refining, preferably a citric acid activated amorphous silica, such as Sorbsil R80 or Trisyl. According to a preferred process the catalyst is neutralised by the addition of a water solution of an acid in a neutralising amount and an adsorbent, and subsequent filtration of the mixture.

According to another preferred process of the invention the purification of the fat composition comprises bleaching to remove polar components and deodorising. When essentially all added water has been removed, for instance by applying a vacuum and slightly increasing the temperature, the reaction mixture is bleached using a traditional oil-bleaching agent such as a bleaching earth or activated carbon. This process is done, for example, by adding 1-3% of a bleaching earth to the neutralised reaction mixture at 90° C. and stirring for 30 minutes. After the bleaching, the slurry containing the precipitated catalyst salt, the adsorbent and the spent bleaching earth is filtrated at a temperature at which the sterol ester formed is still liquid and soluble in the glyceride mixture. In order to remove residual free fatty acids, odour, and flavour components, the product is deodorised using steam distillation. By this process most of the monoglycerides are also removed. The product is heated at a pressure of 100-500 Pa at 150-250° C. and flushed with steam (for example 3% per hour) for 1-3 hours. After cooling, the product is ready for use as an ingredient in the formulation of food, cosmetic or pharmaceutical products.

The invention also refers to a process for the preparation of a fat composition, which after the neutralisation of the catalyst is mixed with a food fat base, such as a fat spread, cheese or shortening fat base, and subsequently purified.

An advantage of the present innovation compared to the conventional way of obtaining sterol/stanol esters in an industrial scale is that an unnecessary step is eliminated by using a direct reaction between the fatty acid base, that is the vegetable oil, and the sterol raw material. In the conventional process the interesterification of the sterol raw material is performed with a fatty acid methyl ester. This process requires as a first step a conversion of a suitable fatty acid base, such as soybean oil, rapeseed oil or any other vegetable or animal oil, having the desired fatty acid composition, into the corresponding methyl ester. This conversion is made by reacting the fatty acid base material with an excess of methanol, using an alkaline interesterification catalyst. Glycerol is liberated in the process and separated from the reaction mixture. Excess methanol is distilled off from the fatty acid methyl ester. This means that large amounts of a potentially hazardous, volatile and inflammable reactant are handled and recycled both in the initial step of methyl ester production and in the subsequent interesterification step. The present innovation eliminates this unnecessary step by utilising a direct reaction between the fatty acid base (vegetable oil/fat) and the sterol raw material.

Another advantage of the process of the present innovation is that it facilitates the use of more sensitive fatty acid bases such as borage oil (rich in gamma-linolenic acid, C18:3 n-6) as well as fish oils (rich in long-chain polyunsaturated fatty acids such as EPA C20:5 n-3 and DHA C22:6 n-3) which are easily oxidised. In the conventional method the unstable polyunsaturated fatty acids are exposed to a larger oxidative stress due to prolonged handling of the methyl ester at elevated temperatures and several processing steps. In the present invention, the conversion to sterol esters takes place in one step which can be run at lower temperatures and shorter times with efficient protection of the product by for example inert gas blanketing.

It is also conceivable that any fatty acid composition can be used in the process according to the present invention. This means that a nutritionally optimised fatty acid composition can be constructed and used as a starting material for the interesterification. Since the fatty acid composition of the sterol esters produced will reflect the starting fatty acid composition, combinations of sterols and fatty acids with nutritionally improved properties will be obtainable. An example of such a combination would be the betasitosterol esters of eicosapentadienoic (EPA, C20:5) and docosahexaenoic (DHA, C22:6) acids obtained from using fish oil as a starting material in the process. In the conventional process the sterol ester fatty acid composition will be determined by the starting material (for example rapeseed oil) and a nutritionally more balanced fatty acid composition will be difficult to achieve.

The invention also refers to a fat composition which can be prepared by the process of the invention, which has a content of, in % by weight of the total composition,

sterol esters 10-95%

free sterols <15%

diglycerides <10%

monoglycerides <1%

triglycerides ad 100%

The fatty acids in the sterol esters and glycerides of the fat composition are selected from the group consisting of C8:0, C10:0, C12:0, C14:0, C16:0, C18:0, C18:1, C18:2, C18:3, C20:5, C22:6.

In the fat composition according to the invention the sterol base is preferably selected from the group consisting of: β-sitosterol, campesterol, β-sitostanol, brassicasterol, stigmasterol, α-amyrin, β-amyrin, cycloartanol, cycloartenol, butyrospermol, lupeol, and methylenecycloartenol.

One major function of the sterol or stanol esters is to lower the serum LDL-cholesterol levels. It is therefore desirable to be able to include phytosterols and phytosterol derivatives in food products in amounts that permit an easy administration of 1-3 g of sterol equivalents per day. The food products in which the sterols are incorporated need to fulfil general nutritional requirements associated with healthy diets as well as being technologically feasible and having good sensory properties.

According to a preferred aspect the invention refers to a food product which comprises a fat composition which can be prepared by the process of the invention, which has a content of 50-75% sterol esters, in % by weight of the fat content of the product.

One preferred way of administering the sterol or stanol is to incorporate them into a margarine/fat spread at a level of approximately 8 g sterol/100 g fat spread. At a typical daily consumption of 20-30 g fat spread per day, this will give a sterol intake within the desired range.

A nutritionally acceptable fat spread product should combine the sterol/stanol material with a low fat content simultaneously with a low overall level of saturated fatty acids. The sensory properties of the finished product as well as its physical appearance, texture and shelf-life also need to be considered. This is done in the present invention by selecting the appropriate triglyceride raw material and triglyceride/sterol ratio. In this respect, the physical properties of the individual sterol/stanol ester are important. For example, beta-sitosteryl oleate prepared by reacting tall oil sitosterol (Ultra Sitosterol, UPM-Kymmene, Lappeenranta, Finland) with oleyl chloride, has a melting point of approximately 40° C. after purification. It is well soluble at room temperature in a vegetable oil blend. A saturated fatty acid sterol/stanol ester has a higher melting point; for example beta-sitostanol palmitates and stearates were reported to melt within the range of 101-105° C. (U.S. Pat. No. 5,892,068). These high melting esters have of course lower solubility in a vegetable oil at room temperature. By optimising the solubility and melting point of the sterol/stanol ester, different textures and consistencies, suitable for different applications, may be obtained.

Food products such as fat spreads, imitation cheeses, bakery shortenings and so on, can be prepared by first mixing the fat composition according to the invention with auxiliary oils and fats to provide a suitable melting profile (solid fat content profile) and nutritional value, while preserving the desired sterol content in the product. A low fat margarine or fat spread can, for example, be produced by mixing the fat composition according to the invention in equal proportions with a hardstock consisting of an interesterified blend of coconut oil and palm oil, and adding an emulsifier (monoglyceride/lecithin) at 60° C. A water phase consisting of water, milk solids, a suitable hydrocolloid (for example gelatine or maltodextrin) and optional flavourings is then emulsified into the oil phase and the resulting water-in-oil emulsion is then subjected to cooling in a scraped-surface heat exchanger.

Imitation dairy products can also be formulated using the fat composition according to the present invention. For example, traditional milk-fat based yoghurt has a fat content of 3% and a typical daily intake could be 1-2 dl (100-200 g). A low-fat yoghurt has a fat content of 0.5%. If the milk-fat is replaced by a fat composition according to the invention having a sterol content of 45%, a typical daily consumption of 200 g would give 0.45 g of sterol if formulated into a low-fat yoghurt and 2.7 g if in a standard product. Other dairy products that can be consumed as such or used as ingredients in cooking, such as cooking cream, sour cream, can also be prepared in a similar way.

A comparable liquid product can be obtained by using unsaturated sterol esters. In the other end of the scale, imitation chocolate bars or filled confectionery products may be formulated using a fat blend according to the present invention with a more saturated sterol/stanol ester. In this context it is also important to consider the properties of the remaining triglycerides and diglycerides which have been obtained in the process.

According to another aspect the invention refers to a cosmetic product containing a fat composition which can be prepared by the process of the invention, which has a content of, in % by weight of the total cosmetic product, 10-30% sterol esters. In many cosmetic applications, it is desirable to have a clear, completely liquid product, for example bath oils. Such products with relatively high sterol contents may be achieved by using unsaturated sterol esters.

The invention also refers to a pharmaceutical product containing in addition to a pharmaceutically active substance a fat composition which can be prepared by the process of the invention, and wherein the content of sterol esters, in % by weight of the fat composition, is 75-90%.

### INTERESTERIFICATION EXAMPLES

In the following examples interesterification reactions between different sterols and different oils were performed. The following methods were used to analyse the starting materials and the final product, that is a fat composition comprising sterol esters, free sterols, and different glycerides, for composition and quality:

Iodine Value

The iodine value was determined according to IUPAC 2.2054, using a modified Hanus method. This will give the unsaturation of the fat composition in mg I₂/g fat.

Hydroxyl Value

The hydroxyl value is defined as the number of mg KOH and was determined according to AOCS Cd 13-60.

Acid Value

The free fatty acid content of the fat composition was determined according to IUPAC 2.201. The acid value is defined as the number of mg of KOH required to neutralize the free fatty acids in 1 g of the fat and is expressed as % of oleic acid.

p-Anisidine Value

This method determines the amount of aldehydes, principally 2-alkenals, in the fat composition in accordance with IUPAC 2.504 and AOCS Cd 18-90 by measuring the absorbance. The aldehydes are oxidation products and a low anisidine value means that the composition is of high quality.

Peroxide Value

The peroxide value is obtained by the method of AOCS Cd 8b-90, which determines all substances in terms of milliequivalents of peroxide per 1000 g of the composition that oxidize KI under the conditions of the test. Said substances are generally assumed to be peroxides or other similar products of fat oxidation.

Solid Content

Solid fat content was determined by the method of IUPAC 2.150 (a) using nuclear magnetic resonance.

Free Sterols

The content of free sterols is calculated by the following method comprising derivatisation and gas chromatography.

1. Silylation

To about 40-50 mg of sample is added 500 μl of an internal standard consisting of 2 mg/ml cholesterol dissolved in chloroform. The solvent is evaporated under nitrogen and 500 μl of MSHFBA is added and then the sample is silylated at 100° C. for 30 minutes. The sample is then diluted with about 1 ml of chloroform.

2. Gas Chromatography

The sample is separated and quantified by gas chromatography on a 15 m DB-1 HT capillary column with SPI injection and temperature programming from 80 to 340° C. The content of free sterols is calculated by means of the internal standard.

Total Sterols

The sterol composition of the sterol raw material and the total sterol content of the final fat composition was determined by means of gas chromatography after derivatisation.

Sterol Esters

The sterol ester content was determined from the total sterol content and the content of free sterols.

Diglycerides

Diglycerides were determined by silylation and gas chromatography.

1. Silylation

To about 40-50 mg of sample is added 500 μl of an internal standard consisting of 6 mg/ml diheptadecanoin dissolved in chloroform. The solvent is evaporated and 500 μl of the silylation agent MSHFBA is added. The sample is then silylated at 100° C. for 30 minutes.

2. Gas Chromatography

After the silylation the sample is diluted with an appropriate volume of chloroform, that is 2-5 ml, and gas chromatographied on a 15 m DB-1 HT capillary column with SPI injection and temperature programming from 80 to 340° C. The diglycerides are identified and quantified against the added internal standard.

### Example 1

### Interesterification of Sitosterols with Canola Oil

a) A mixture of 270 g (45% w/w) Sitosterol Ultra (UPM Kymmene, Lappeenranta, Finland) and 330 g (55% w/w) of low erucic acid rapeseed oil, that is canola oil (Karlshamns AB, Karlshamri, Sweden) were dried for 45 minutes at 140° C. in vacuum in a standard glass processing vessel. The sterol material has the following composition: beta-sitosterol 90-92% (including beta-sitostanol 12-15%), campesterol 5-6% (including 0.2-0.5% campestanol), alpha-sitosterol 0-1% and other unspecified sterols 2-3%. The vessel contents were flushed with nitrogen and 0.6 g (0.1%) sodium methylate (Huls AG, Marl, Germany) was added. The interesterification reaction was carried out at 140° C. for 180 minutes. After cooling to 70° C., 6 g (1%) of a 20% solution of citric acid (anhydrous, ADM Ringaskiddy, Co Cork, IRL) and 1.2 g (0.2%) of Sorbsil R80 (Crosfield, Warrington, Cheshire, UK) were added. The mixture was flushed with nitrogen for 15 minutes at 70° C., then vacuum was applied and the temperature was increased to 90° C. The reaction was bleached using 12 g (2%) of Tonsil Optimum 215 FF (SüdChemie, München, Germany) at 90° C. for 30 minutes. The product was cooled to 65° C. and filtered over a paper filter. The product was then deodorised at −30° C. for 120 minutes at 100-500 Pa using 3% (w/w) water vapour per hour. The final product, a yellow viscous liquid, was analysed for composition and quality.

b) The process described under a) was repeated using 30 g (5% w/w) of Ultra Sitosterol to 570 g (95% w/w) of low erucic acid rapeseed (canola) oil. The reaction was carried out at 120° C. for 180 minutes using 0.1% sodium methylate as catalyst. The reaction mixture was neutralised using 1% of a 20% citric acid solution at 90° C. with nitrogen flushing for 10 minutes, then 30 minutes in vacuum and bleached with 2% Tonsil Optimum 215 FF at 90° C. for 30 minutes. After filtering at 65° C. the product was deodorised at 230° C. for 120 minutes with 3% water vapour per hour.

c) In a similar manner 150 g (25% w/w) Ultra Sitosterol was reacted with 450 g (75% w/w) of canola oil. This reaction time was 180 minutes at 130° C.; other conditions being as above.

The products obtained had the following characteristics:

| | a | b | c |
|---|---|---|---|
| Iodine value | 96 | 111 | 103 |
| Hydroxyl value | 21 | 8 | 12 |
| Free fatty acids | 0.02 | <0.01 | 0.02 |
| Anisidine value | 0.4 | 0.2 | 0.3 |
| Peroxide value | 0.1 | 0.2 | <0.1 |
| Solid fat content | | | |
| @ 10° C. | 23% | <0.5 | 6 |
| @ 20° C. | 5% | <0.5 | 2 |
| @ 30° C. | 2% | 0 | <0.5 |
| @ 35° C. | 1% | 0 | <0.5 |
| @ 40° C. | 0% | 0 | 0 |

The composition of the final products were as follows

| | a | b | c |
|---|---|---|---|
| Sterol esters (%) | 66.0 | 9.7 | 37.5 |
| Free sterols (%) | 6.6 | 0.2 | 1.4 |
| Monoglycerides (%) | 0.5 | 0.1 | 0.3 |
| Diglycerides (%) | 7.1 | 5.7 | 7.3 |
| Triglycerides (%) | 16.9 | 81.3 | 44.8 |
| Unidentified (%) | 2.9 | 3.0 | 8.5 |

### Example 2

### Interesterification of Sitosterol with Different Oils

The interesterification reaction was carried out as described in Example 1 using Ultra Sitosterol and the following oils and conditions:

a) 120 g (20% w/w) of sitosterol and 480 g (80% w/w) of borage oil (Karlshamns AB, Karlshamn, Sweden) were heated at 130° C. The borage oil is characterised by the following fatty acid composition: C16:0 9%, C18:0 2%, C18:1 15%, C18:2 40%, gamma-C18:3 26%, C20:1 4%, others 4%.

b) 180 g (30% w/w) or sitosterol and 420 g (70% w/w) of fish oil (Ropufa 30, Roche Vitamins, Basel, Switzerland) were heated at 120° C. The fish oil was characterised by the following fatty acid composition: C14:0 5%, C16:0 15%, C16:1 8%, C18:0 3%, C18:1 12%, C18:4 4%, C20:1 3%, C20:5 12%, C22:1 5%, C22:6 18%, others 15%.

c) 240 g (40% w/w) of sitosterol and 360 g (60% w/w) of palm mid fraction (PMF, Karlshamns AB, Karlshamn, Sweden) were heated at 140° C. The palm mid fraction was characterised by the following fatty acid composition: C16:0 51%, C18:0 5%, C18:1 37%, C18:2 6%, others 1%.

d) 330 g (55%) of sitosterol and 270 g (45) of coconut oil CNO, Karlshamns AB, Karlshamn, SE) were heated at 140° C. The coconut oil is characterised by the following fatty acid composition: C8:0 9%, C10:0 6%, C12:0 47%, C14:0 18%, C16:0 9%, C18:0 3%, C18:1 6%, C18:2 2%.

The products obtained had the following characteristics:

| | a | b | c | d |
|---|---|---|---|---|
| Iodine value | 135 | 140 | 61 | n.a. |
| Hydroxyl value | 25 | 26 | 23 | 19 |
| Free fatty acids | 0.02 | 0.02 | <0.01 | <0.01 |
| Anisidine value | 5.5 | 1.6 | 0.7 | 0.1 |
| Peroxide value | 0.6 | <0.1 | <0.1 | 0.6 |
| Solid fat content | | | | |
| @ 10° C. | 9 | 14 | 50 | 82 |
| @ 20° C. | 9 | 11 | 40 | 64 |
| @ 30° C. | 8 | 9 | 35 | 49 |
| @ 35° C. | 7 | 7 | 32 | 43 |
| @ 40° C. | 7 | 6 | 30 | 38 |
| Free sterols (%) | 12.4 | 2.8 | n.a. | 8.2 |

The upper limit of sterol inclusion in a vegetable oil according to the invention can be calculated from the stoichiometry of the reaction. It can be assumed that the upper practical limit of free sterols in the product is 10% w/w and that all glycerides added are converted into sterol esters. Beta-sitosterol has a molecular weight of 415 g/mol, rapeseed oil has an average molecular weight of 883 g/mol and coconut oil has an average molecular weight of 680 g/mol, rapeseed oil and coconut oil representing a high and low molecular weight triglyceride source, respectively. At the weight ratios of 62/38 (sterol/rapeseed oil) and 68/32 (sterol/coconut oil) in the starting material, all glycerides have been converted into sterol esters and the maximum amount of 10% of free sterols is obtained owing to the state of equilibrium.

### Example 3

### Interesterification of Different Sterol Materials with Canola Oil

Different sterol raw materials were interesterified with canola oil.

a) Canola oil (510 g, 85% w/w) and soya sterols (Generol 122N, Cognis, Germany) (90 g, 15% w/w) were dried at 130° C. for 30 minutes and interesterified at 130° C. for 180 minutes using 0.1% Na-methylate. Workup consisting of neutralisation, bleaching, filtering and deodorisation was performed as described in Example 1. The composition of the sterol raw material was analysed to be as follows: beta-sitosterol 48%, campesterol 26%, stigmasterol 18%, delta-5-avenasterol 1%, others 7%.

b) In a similar manner, canola oil (390 g, 65% w/w) and canola sterols (Generol R, Cognis, Germany) (210 g, 35% w/w) were interesterified at 140° C. for 180 minutes. The sterol raw material had the following characteristics: total sterols 90-100%, sitosterol 40-60%, campesterol 30-45%, brassicasterol 8-18%.

The products obtained had the following characteristics:

| | a | b |
|---|---|---|
| Iodine value | 111 | 107 |
| Hydroxyl value | 10 | 26 |
| Free fatty acids | 0.01 | 0.02 |
| Anisidine value | 0.9 | 1.3 |
| Peroxide value | 0.7 | 0.4 |
| Solid fat content | | |
| @ 10° C. | 1 | 8 |
| @ 20° C. | <0.5 | 3 |
| @ 30° C. | <0.5 | 1.5 |
| @ 35° C. | 0 | <0.5 |
| @ 40° C. | 0 | <0.5 |

### USE EXAMPLES

In the following Examples 5-7 the product obtained in Example 1a) is used for the manufacture of different food products. In Examples 8-9 the products from Example 2, a) and b) respectively, are used for making cosmetic products.

### Example 4

### Fat Spread

Low fat spreads were prepared on a pilot plant margarine crystalliser (Armfield FT25BBP, Armfield Ltd, Ringwood, Hampshire, England). 49 parts of the fat composition according to Example 1a was mixed with 51 parts of a standard fat spread non-trans fat spread hardstock based on interesterified coconut oil/palm oil to produce a fat spread fat phase MFP1. This fat phase had the following melting profile: SFC (10 C)=26%, SFC (20 C)=14%, SFC (30 C)=3%, SFC (35 C)=<2%, SFC (40 C)=<2% (SFC=Solid Fat Content as measured by low-resolution pulsed NMR using the IUPAC 2.150).

The fat spread was prepared according to the following recipe:

Oil Phase:

| | |
|---|---|
| fat spread fat phase MFP1 | 39.29% |
| emulsifier (saturated monoglyceride/lecithin 1:1) | 0.70% |
| colouring (beta-carotene, 30% in oil) | 0.00167% |
| flavouring | q.s. |

Aqueous Phase:

| | |
|---|---|
| water | 54.3% |
| skim milk powder | 1.0% |
| salt (NaCl) | 1.6% |
| gelatine (230 bloom) | 3.0% |
| potassium sorbate | 0.1% |
| citric acid to pH = 5.8. | |

The oil phase was melted and mixed at 60° C. The aqueous phase was mixed at 60° C. and added slowly whilst stirring to the oil phase. Crystallisation was carried out in the pilot crystalliser using a barrel-barrel-pin worker configuration with a final product temperature of 14° C. The fat spread product will give a daily dose of 1.4-2.1 g sterol when consumed in normal amounts (20-30 g of fat spread per day).

### Example 5

### Imitation Yoghurt

An imitation yoghurt product was prepared by the following procedure: 1000 g of skimmed milk powder and 1000 g of sucrose were dissolved in 8000 g of water (aqueous phase). 170 g of product according to Example 1a was mixed with 20 g of an interesterified margarine basestock and 10 g of anhydrous milk fat at 60° C. 5 g of an emulsifier mixture (distilled saturated monoglyceride:soybean lecithin 2:1) was added and the mixing was continued until the emulsifier was dissolved (oil phase). 200 g of the oil phase was emulsified into 9800 g of the water phase using a high-speed mixer. After homogenisation and pasteurisation, the emulsion was fermented using a conventional Lactobacillus delbrueckii spp. bulgaricus and Streptococcus salivarius spp thermophilus starter culture at 42-45° C. The resulting product had a fat content of 2% and will give 1.1 g of sterols per 150 g serving.

### Example 6

### Sour Cream

A sour cream (“creme fraiche”) was produced according to the following procedure: 500 g skimmed milk powder and 15 g sugar were dissolved in 4885 g of water at 60-70° C. to produce an aqueous phase. An oil phase containing 3600 g of product according to Example 1a, and 3 g of a distilled monoglyceride (Dimodan RT from Danisco Food Ingredients, Brabrand, DK) was mixed and heated to 60-70° C. The oil phase was emulsified into the water phase using a high speed mixer and homogenised in a Panda-NS 1001L homogeniser (Niro-Soavi SpA, Parma, IT) at 120 bar pressure at 60° C. 9 kg of this vegetable oil emulsion was mixed with 1 kg dairy cream (fat content 40%) and the resulting mixed cream was pasteurised in an Armfield FT 47 pasteuriser at 120° C. and cooled to 21° C. 20 ml of a starter culture (CH-N 11 freeze-dried Lactic Culture for Direct vat Set, 50 units in 500 ml milk, from Chr Hansen, Copenhagen, Denmark) was added to the pasteurised emulsion and incubated at 21° C. for approximately 24 hours to pH=4.5. A daily serving of 10 g of this product will give an intake of 1.8 g of sterol.

### Example 7

### Cooking Cream

An all-round use cooking cream with a fat content of 15% and a sterol content of 3% was prepared by the following procedure: 560 g of skimmed milk powder, 100 g of Grindsted FF3113 (emulsifier and stabiliser, Danisco Ingredients, Brabrand, Denmark) were mixed and dissolved in 7840 g of cold water. The aqueous phase was heated to 60° C. 750 g of product according to Example 1a was mixed with 750 g of AKOBLEND (hydrogenated vegetable fat, Karlshamns AB, Karlshamn, Sweden), heated to 60° C. and emulsified into the water phase using a high speed mixer. The emulsion was homogenised at 60° C. and 50 bar and pasteurised at 120° C. This imitation cooking cream can be used as a base for producing sauces, toppings, soups and other suitable food items.

### Example 8

### Ointment Cream Base

An ointment cream base with moisturising and protective properties was prepared in the following way:

| | INCI name | % w/w |
|---|---|---|
| Phase A | | |
| AKOGEL | Hydrogenated vegetable | 9.0 |
| | oil | |
| Product from Example 2a | | 5.0 |
| AKOMED R | Caprylic/capric | 2.5 |
| | triglycerides | |
| Eutanol G | Octyldodecanol | 1.5 |
| Cetyl alcohol | Cetyl alcohol | 2.5 |
| Distilled saturated | Glyceryl stearate | 2.4 |
| monoglycerides | | |
| Eumulgin B1 | Ceteareth-12 | 1.2 |
| Eumulgin B2 | Ceteareth-20 | 0.8 |
| Stearic acid | Stearic acid | 0.1 |
| Phase B | | |
| Glycerol (99.5%) | Glycerine | 3.0 |
| KOH (1% in water) | | 2.6 |
| Water | | 69.4 |

Heat phases A and B separately to 75° C. Add phase A slowly to phase B while stirring. Cool to 55° C. and homogenise. Adjust pH using citric acid to 6.0, cool to 35° C. and add preservative and perfume. Cool to room temperature.

The obtained product contains approximately 0.5% of sterol esters.

### Example 9

### Body Care Lotion

A protective body care lotion with moisturising properties was prepared in the following way:

| | INCI Name | % w/w |
|---|---|---|
| Phase A | | |
| Arlatone 985 | Polyoxyethylene stearyl stearate | 4.0 |
| Brij 721 | Steareth-21 | 2.0 |
| Eutanol G | Octyldodecanol | 5.0 |
| AKOGEL | Hydrogenated vegetable oil | 5.0 |
| Product from Example | | 5.0 |
| 2b | | |
| Phase B | | |
| Atlas G-2330 | Sorbeth-30 | 2.5 |
| Water | | 76.0 |
| Phenonip | Esters of p-hydroxybenzoic acid | 0.5 |
| | (preservative) | |

Heat phases A and phase B separately to 75° C. while stirring. Add phase A slowly to phase B while stirring. Cool to 55° C. and homogenise using a high-speed mixer or a valve homogeniser. Cool to room temperature.

The product obtained contains approximately 1.9% of sterol esters and 0.1% of free sterols.

1-18. (canceled)

19. A method for preparing a fat composition comprising
reacting a sterol raw material with a triglyceride raw material to create a fat composition; and
isolating the fat composition;
wherein the ratio of the sterol raw material to the triglyceride raw material in the mixture is from 5/95 to 65/35; and
wherein the fat composition has a content of, in % by weight of the total composition,
sterol esters 10-95%
free sterols <15%
diglycerides <10%
monoglycerides <1%
triglycerides ad 100%.

20. The method of claim 19, wherein the reacting is conducted at a temperature of from 100° C.-200° C.

21. The method of claim 19, wherein the reacting is conducted at a temperature of from 100° C.-140° C.

22. The method of claim 19, wherein the reacting is conducted for a time of from 60 minutes-180 minutes.

23. The method of claim 19, wherein the reacting further comprises an alkaline catalyst.

24. The method of claim 23, wherein the alkaline catalyst is selected from the group consisting of sodium hydroxide, potassium hydroxide, sodium methoxide, sodium ethoxide, sodium glycerolate, and mixtures thereof.

25. The method of claim 24, wherein the isolating comprises neutralizing the alkaline catalyst by addition of a water solution comprising an acid in a neutralizing amount.

26. The method of claim 25, further comprising
adding an absorbent to create a blend; and
filtering the blend.

27. The method of claim 19, wherein the content of free sterols in the fat composition is <10%.

28. The method of claim 19, wherein the content of sterol esters, in % by weight of the fat composition, is from 50-75%.

29. The method of claim 19, wherein the triglyceride raw material comprises fatty acids selected from the group consisting of C8:0, C10:0, C12:0, C14:0, C16:0, C18:0, C18:1, C18:2, C18:3, C20:5, and C22:6.

30. The method of claim 19, wherein the triglyceride raw material comprises glycerol esters of saturated or unsaturated C4-C28 fatty acids.

31. The method of claim 19, wherein the triglyceride raw material comprises glycerol esters of saturated or unsaturated C16-C22 fatty acids.

32. The method of claim 19, wherein the triglyceride raw material comprises at least one member selected from the group consisting of rapeseed oil, crambe oil, mustard seed oil, cottonseed oil, peanut oil, linseed oil, grapeseed oil, safflower oil, sesame oil, tea seed oil, corn fibre oil, corn bran oil, wheat oil, wheat bran oil, wheat germ oil, oat oil, oat bran oil, rice bran oil, rice oil, palm kernel oil, babassu oil, illipe butter, Borneo tallow, shea butter, shea oil, madhuca, mowrah butter, sal butter, avocado oil, avocado seed oil, cocoa butter, hazelnut oil, almond oil, macadamia nut oil, walnut oil, canola oil, corn oil, peanut oil, soybean oil, sunflower oil, palm oil, coconut oil, borage oil, evening primrose oil, blackcurrant seed oil, fish oil, linseed oil, and mixtures thereof.

33. The method of claim 19, wherein the triglyceride raw material comprises at least one member selected from the group consisting of rapeseed oil, canola oil, corn oil, peanut oil, soybean oil, sunflower oil, palm oil, coconut oil, borage oil, evening primrose oil, blackcurrant seed oil, fish oil, linseed oil, and mixtures thereof.

34. The method of claim 19, wherein the sterol raw material comprises >90% by weight of one or more alcohols selected from the group consisting of β-sitosterol, β-sitostanol, campesterol, campestanol, brassicasterol, and mixtures thereof.

35. The method of claim 19, wherein the sterol raw material comprises at least one member selected from the group consisting of tall oil sterols, partially hydrogenated tall oil sterols, completely hydrogenated tall oil sterols, soybean sterols, partially hydrogenated soybean sterols, hydrogenated soybean sterols, rapeseed sterols, partially hydrogenated rapeseed sterols, hydrogenated rapeseed sterols, canola sterols, partially hydrogenated canola sterols, hydrogenated sterols, lobra sterols, partially hydrogenated lobra sterols, hydrogenated lobra sterols, sunflower sterols, partially hydrogenated sunflower sterols, hydrogenated sunflower sterols, and mixtures thereof.

36. The method of claim 19, wherein the sterol raw material comprises of at least one alcohol selected from the group consisting of β-sitosterol, campesterol, β-sitostanol, brassicasterol, stigmasterol, α-amyrin, β-amyrin, cycloartanol, cycloartenol, butyrospermol, lupeol, methylenecycloartenol, and mixtures thereof.

37. The method of claim 19, wherein the sterol raw material comprises at least one alcohol selected from the group consisting of β-sitosterol, β-sitostanol, campesterol, campestanol, brassicasterol, and mixtures thereof.
